(19) United States
(12) Plant Patent Application Publication
(10) Pub. No.: US 2002/0059666 P1
(43) Pub. Date: May 16, 2002

(54) Poinsettia plant named 'Fislemon'

(75) Inventor: Katharina Zerr, Hohr-Grenzhausen (DE)

Correspondence Address: FOLEY & LARDNER, Suite 500, 3000 K Street, N.W., Washington, DC, 20007-5109, US

(73) Assignee: FLORFIS AG

(21) Appl. No.: 09/850,076

(22) Filed: May 8, 2001

(30) Foreign Application Priority Data
Nov. 11, 2000 (EP) 2000/1674

(51) Int. Cl. A01H 5/00

(52) U.S. Cl. PLT/305

(57) ABSTRACT

A new and distinct cultivar of Poinsettia plant named ‘Fislemon’, as described and illustrated, and particularly characterized by the combined features of light yellow, uniform bract color; medium sized inflorescence with weakly lobed bracts; uniform dark-green, distinctly lobed foliage; medium sized plant habit, well-branched and relatively wide; and early flowering response.

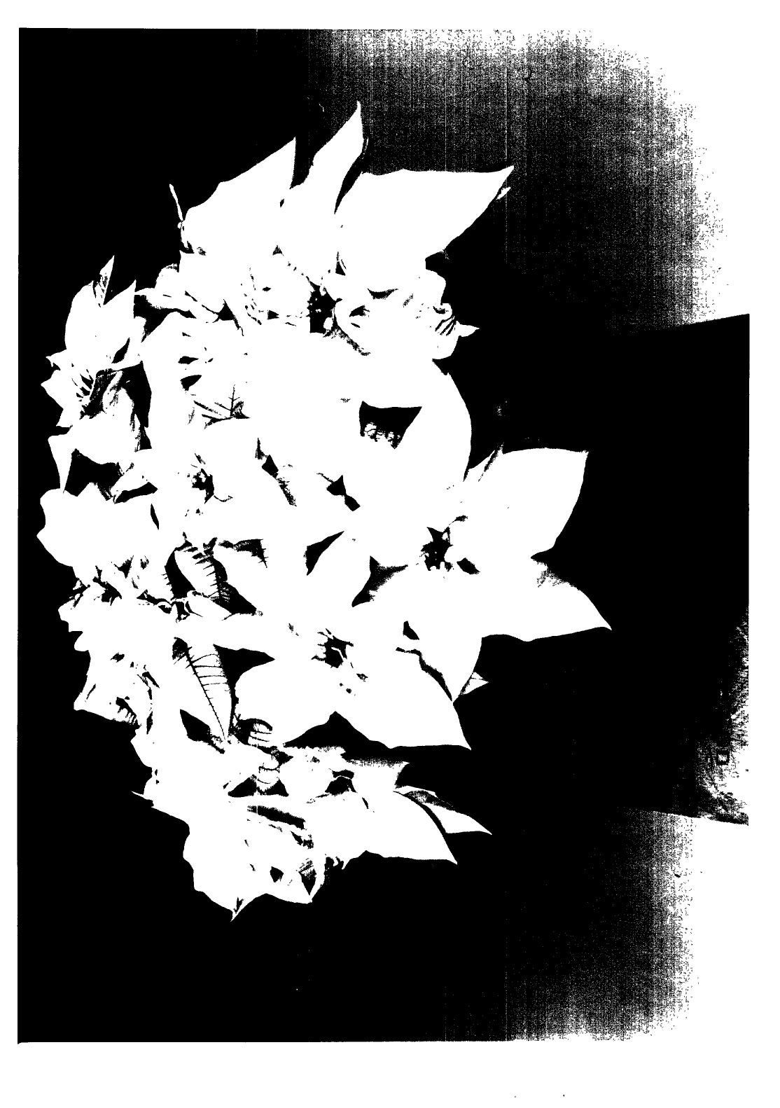

### BACKGROUND OF THE INVENTION

The present invention relates to a new and distinct cultivar of Poinsettia plant known by the cultivar name ‘Fislemon’, and botanically known as Euphorbia pulcherrima.

‘Fislemon’ is a product of a planned breeding program which had the objective of creating new Poinsettia cultivars with white or yellow flower color in combination with dark-green foliage and good cultivation ability.

‘Fislemon’ originated from a hybridization made by the inventor, Katharina Zerr, in Hillscheid, Germany, in 1996. The female parent was a proprietary hybrid seedling, no. S90-602-1, characterized by salmon-pink bract color, medium green foliage, and medium sized, wide and bushy plant habit. The male parent was a hybrid seedling, no. S90-502-1, having red bract color, dark green foliage, and early flowering.

The seeds germinated in the spring of 1997, and the resulting seedlings were identified by numbers. In the summer of 1997, a cutting was taken from each seedling, rooted and grown out for examination as flowering single-stem plants. ‘Fislemon’ was discovered and selected as one flowering plant no. 6935 within the progeny of the stated cross/parentage by Katharina Zerr in the fall of 1997. After this plant had been chosen, more cuttings were taken from the original seedling and grafted on rootstocks of the variety ‘Beckmann's Altrosa’ (U.S. Plant Pat. No. 9,336), in order to transmit the branching causing agent, phytoplasma, into the clone to improve the branching causing characteristics. From the upper area of the successfully grafted plants, shoot tip cuttings were taken for the cultivation of branched plants for the second examination in the fall of 1998.

Horticultural examination initiated in the fall of 1999 and continuing thereafter, has demonstrated that the combination of characteristics as herein disclosed for ‘Fislemon’ are firmly fixed and are retained through successive generations of asexual reproduction. The new cultivar reproduces true to type.

### BRIEF DESCRIPTION OF THE INVENTION

The following traits have been repeatedly observed and are determined to be basic characteristics of ‘Fislemon’ which in combination distinguish this Poinsettia as a new and distinct cultivar:

1 . light yellow, uniform bract color;

2. medium sized inflorescence with weakly lobed bracts;

3. uniform dark-green, distinctly lobed foliage;

4. medium sized plant habit, well-branched and relatively wide; and

5. early flowering response.

‘Fislemon’ has not been observed under all possible environmental conditions. The phenotype may vary significantly with variations in environment such as temperature, light intensity, and day length.

The following observations, measurements and comparisons describe plants grown in Hillscheid, Germany, under greenhouse conditions which approximate those generally used in commercial practice.

Of the may commercial cultivars known to the inventor, the most similar in comparison to ‘Fislemon’ are the variety ‘White Freedom’ and the commercial, unpatented cultivar ‘Lemon Drop’.

In contrast to the cream-colored bracts of ‘White Freedom’, ‘Fislemon’ has light-yellow colored bracts. The shape of the bracts is similar, but those of ‘Fislemon’ appear less ‘heavy’ as they are smaller in size, less rugose, and horizontally or slightly upward directed, not overhanging. Additionally, ‘Fislemon’ does not start flowering quite as early.

In comparison to ‘Lemon Drop’, ‘Fislemon’ has a lighter tone of bract color, RHS 4 C compared to RHS 5 D for ‘Lemon Drop’, and slightly longer bracts with a less smooth/less flat surface. Furthermore, ‘Fislemon’ has a wider plant habit, which enables almost all branches to develop complete, full-grown inflorescence, while the plant habit of ‘Lemon Drop’ is narrower and more upright with about one third of the initially formed branches not really growing out.

### BRIEF DESCRIPTION OF THE DRAWING

The accompanying color photographic drawing shows typical inflorescence and foliage of ‘Fislemon’, with colors being as true as possible with illustrations of this type. The photograph shows a mature potted plant.

### DETAILED BOTANICAL DESCRIPTION

The plants described were grown in a greenhouse in Hillscheid, Germany, in the fall of 1999. Rooted cuttings were planted into 14 cm pots on July 23, and were pinched 12 days after that, leaving 8 nodes. The minimum temperature was about 20° C. until the end of September, and about 17-18° C. thereafter. The plants initiated flowers under natural short-day conditions in fall.

Observations and measurements were mainly taken at the beginning of full flowering. In the following description color references are made to The Royal Horticultural Society Colour Chart (RHS). The color values were determined indoors in a north light.

Classification:

Botanical.—Euphorbia pulcherrima

Commercial.—Poinsettia, cv. ‘FISLEMON’

Parentage:

Female parent.—Hybrid seedling no. K90-602-1

Male parent.—Hybrid seedling no. K90-502-1

Plant:

Form.—Shrub, self-branching

Growth habit.—Moderately compact, medium height pinched plants are bushy and wide in shape

Height (above soil line).—32.5 cm

Width.—55 cm

Average number of branches.—7-9

Stem color.—Light green, RHS 143 C

Internodes.—35 mm

Rooting.—Fast to medium, less than 20 days

Blooming habit.—Begin under natural short day conditions in fall; botanically (cyathia open) in early December; commercially (bracts colored, marketable) in late November

Flowering response time.—about 8.5 weeks

Flowering season.—Up to 5 weeks or longer

Lasting quality (shelf life).—Relatively good, about 28 days

Foliage:

Shape.—Ovate, with straight base, medium expression of lobes, and with acute to acuminate tip

Margin.—Entire

Upper leaf surface.—Smooth and flat texture; only weakly veined, color of veins; RHS 145 A

Lower leaf surface.—Flat and smooth texture, except for the slightly protruding midrib and finer side veins, in a palmate pattern, the vein color is greenish-white, RHS 145 C

Leaf blade size.—Length 14.0 cm Width 10.2 cm

Color: Uniform, dark-green green

Mature foliage.—Upper surface RHS 139 A; lower surface, RHS 137 A

New foliage.—Upper surface RHS 144 A to 143 A; lower surface RHS 144 A

Leaf petiole.—Upper surface light green RHS 145 B, no anthocyanin; lower surface greenish-white RHS 145 C

Petiole: Length 7.5 cm

Aspect: The petioles are horizontally directed, with the leaf blades horizontally directed or slightly downwards

Disease Resistance: No special observations made

Flowering Description:

Whole inflorescence with surrounding bracts: medium sized, mainly horizontally directed, young bracts slightly upward directed

Diameter.—About 26 cm

Number of bracts per inflorescence.—9-11 (length over 2 cm)

Size of largest bracts.—12.3 cm long, 9.3 cm wide

Bract shape.—Ovate, with an almost straight base, only weak lobes, and with acute to acuminate tip

Surface texture.—Almost flat and slightly rugose(increases with maturing of bracts); the veins are only weakly expressed on the surface, their color corresponds closely to the bract color, the base of the midrib is about RHS 145 B; the veins on the lower surface are slightly protruding, their color is a light, yellowish-green, approximately RHS 150 C, and the base of the midrib RHS 145 A to RHS 145 B, light green.

Color.—Generally a light lemon-yellow, uniform; upper surface RHS 4 C lower surface RHS 154 D

Petiole.—Length: 2.0 cm, light green color RHS 145 B

Cyme.—About 10-15 cyathia, in a narrow cluster, about 20 mm wide

Cyathium.—About 50 mm wide, light grass-green color RHS 143 A to 143 C, the top is a pale yellow, RHS 4 C to RHS 4 D Nectar Cups: about 50 mm wide, intense yellow, RHS 7 A, no anthocyanin

Reproductive Organs:

Stamens.—very light yellow, RHS 4 C

Pollen.—yellow, plenty

Pistils.—Style and stigma color very light yellow, RHS 4 C

Ovaries.—Medium green, RHS 143 A, triangular, 3 ovules

Seed set: Under the greenhouse conditions described no spontaneous seed set was observed, but fertile when pollinated, shape and development are typical for the species

I claim:

1. A new and distinct cultivar of Poinsettia plant named ‘Fislemon’, as described and illustrated herein.
